(19) United States
(12) Patent Application Publication
(10) Pub. No.: US 2012/0197221 A1
(43) Pub. Date: Aug. 2, 2012

(54) TRANSEPIDERMAL DRUG DELIVERY SYSTEM CONTAINING RIVASTIGMINE

(76) Inventors: Myoung Hwa JANG, Seongnam-Si (KR); Dong Il Lee, Seongnam-si (KR); Mun Seok Han, Suwon-si (KR); Tae Wan Lee, Yongin-si (KR); Jong Kyoo Lee, Seoul (KR); Han Ki Kim, Yongin-si (KR)

(21) Appl. No.: 13/324,858

(22) Filed: Dec. 13, 2011

(30) Foreign Application Priority Data
Jan. 28, 2011 (KR) 10-2011-0008531

(51) Int. Cl. A61K 31/27; A61M 35/00; A61P 25/16; A61K 9/70; A61P 25/28

(52) U.S. Cl. 604/307; 424/443; 514/490

(57) ABSTRACT

Disclosed is a pharmaceutical composition containing rivastigmine. Specifically, disclosed is a transepidermal drug delivery system including a rivastigmine-containing drug layer and a supporter adhered to one surface of the drug layer to support the drug layer, wherein the drug layer contains 10 to 40 parts by weight of a rubber, 20 to 80 parts by weight of a rosin ester resin and 0.1 to 10 parts by weight of an acrylic adhesive and the drug layer has a thickness of 40 μm to 100 μm.

This application claims the benefit of Korean Patent Application No. 10-2011-0008531, filed on Jan. 28, 2011, which is hereby incorporated by reference as if fully set forth herein.

### BACKGROUND OF THE INVENTION

1. Field of the Invention

The present invention relates to a pharmaceutical composition containing rivastigmine. More specifically, the present invention relates to a transepidermal drug delivery system containing rivastigmine which inhibits progression of dementia such as Alzheimer's disease and Parkinson's disease.

2. Discussion of the Related Art

Rivastigmine [(S)—N-ethyl-N-methyl-3-[1-(dimethylamino)ethyl]phenyl-carbamate] represented by the following Formula I inhibits activities of acetylcholine sterase (AChE) and butylcholine sterase (BuChE) in plaques and tangles and thus suppresses propagation of dementia such as Alzheimer's disease and Parkinson's disease.

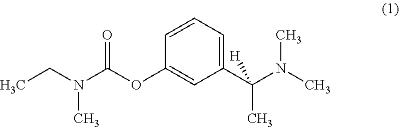

A transepidermal drug delivery system in which a drug is absorbed through the skin can continuously deliver the drug at a constant level to the body for a long period of time and prevent initial metabolism in the liver.

In particular, the transepidermal drug delivery system is known as a method which can efficiently decrease the frequency of drug administration and reduce side effects due to superior drug compliance of patients. However, most drugs are physically and chemically unsuitable for application to transepidermal drug delivery systems and cannot secure safety to the body, and the very few drugs can be as applied to transepidermal drug delivery systems, since the skin has considerably low permeability.

Meanwhile, dementia is a senile disorder in modern society, which constitutes a serious human health problem, a result of the rapidly increasing portion of the population who are elderly. The first anti-dementia medication came on the market in 1993. At present, novel dementia drugs are actively developed by multinational corporations. Research associated with dementia medicines has a short history, as compared to other medicines and development of methods for treating the root causes of dementia as well as transepidermal drug delivery systems containing a dementia drug is highly attractive to the pharmaceutical industry.

WO 2007/064407 (hereinafter, referred to as “D1”) discloses a transdermal therapeutic system having a silicone adhesive layer to provide a specific plasma concentration. The transdermal therapeutic system of D1 contains an Alzheimer's disease-type dementia drug such as rivastigmine and donepezil, a silicon polymer and an adhesive. The transdermal therapeutic system of D1 provides a technique in which the silicone adhesive layer has not an effluence on permeation of the active ingredient (medicinal component) in the skin and improves adhesion. However, the transdermal therapeutic system of D1 has a four-layer structure which includes a drug layer containing an active ingredient, a silicone adhesive layer, a supporter and a release film, thus disadvantageously entailing a plurality of manufacturing processes.

WO 2007/129427 (hereinafter, referred to as “D2”) discloses a transepidermal drug delivery system to stably administer an anti-dementia drug for a long period of time. However, the transepidermal drug delivery system of D2 has a five-layer structure which includes an adhesive layer containing an acrylic adhesive, a control film to control permeation of the drug in the skin, a reservoir layer containing a high amount of anti-dementia drug, a supporter and a release film, thus disadvantageously entailing a plurality of manufacturing processes.

Korean Patent Laid-open No. 10-2010-0080681 (hereinafter, referred to as “D3”) discloses a transepidermal drug delivery system containing rivastigmine as an anti-dementia drug and a salt thereof as an active ingredient. The transepidermal drug delivery system of D3 has a two-layer structure which includes a supporter and an adhesive layer containing a drug and can thus be manufactured through a simple process, and contains a transdermal absorption accelerator, thus exhibiting superior transdermal absorptivity of rivastigmine. However, the transdermal absorption accelerator disadvantageously stimulates the skin tissue while the drug permeates into the skin which has considerably low permeability although it is a component which is harmless to the body.

### SUMMARY OF THE INVENTION

Accordingly, the present invention is directed to a pharmaceutical composition containing rivastigmine that substantially obviates one or more problems due to limitations and disadvantages of the related art.

The transepidermal drug delivery system containing rivastigmine should exhibit designed transdermal absorptivity, be harmless (safe) to the skin and exhibit superior adhesivity. Accordingly, pharmaceutical research in which the transepidermal drug delivery system should be composed of all components which are harmless (nontoxic) to the human and the active ingredient is safely absorbed in the skin without causing damage to the skin by release of the active ingredient and permeation of the drug into the skin is a core technique. In addition, the transdermal absorption accelerator cannot be completely free of skin stimuli although it has superior human safety. The transepidermal drug delivery system containing no transdermal absorption accelerator has been required in the art, but development of transepidermal drug delivery systems which exhibit a desired drug release rate without any transdermal absorption accelerator is a considerably difficult technique which should be solved.

It is one object of the present to provide a transepidermal drug delivery system for treating Alzheimer's disease and Parkinson's disease-type dementia which exhibits drug dissolution and skin permeation effects, comparable to Exelon Patch, as a control drug, (trade name, manufactured by Lohmann Therapie-Systeme, AG; referred to as ‘control drug E’), is safe to the skin, exhibits superior adhesivity, and is prepared in a simpler manner than the control drug E.

During research on a transepidermal drug delivery system compositions containing rivastigmine, the present inventors discovered that, when a drug layer consisting of a rubber and an adhesive resin further contains an acrylic adhesive, the drug layer exhibits superior adhesivity and drug release and skin permeation effects, comparable to the control drug E. The present invention has been completed based on this discovery.

In accordance with one aspect of the present invention, provided is a transepidermal drug delivery system including a rivastigmine-containing drug layer; and a supporter adhered to one surface of the drug layer to support the drug layer, wherein the drug layer contains 10 to 40 parts by weight of a rubber, 20 to 80 parts by weight of a rosin ester resin and 0.1 to 10 parts by weight of an acrylic adhesive, and the drug layer has a thickness of 40 μm to 100 μm.

As used herein, the term “rivastigmine” includes rivastigmine and any salts thereof. The term “parts by weight” may be substituted by “% by weight”. Specifically, assuming that the total content of components present in the drug layer is 100 parts by weight, the content of respective components may be represented as % by weight. In this case, the contents of respective components may be represented based on dry weight of the drug layer.

Preferably, the transepidermal drug delivery system of the present invention may have a structure including three layers, i.e., a release film, a drug layer and a supporter.

In accordance with another aspect of the present invention, provided is a transepidermal drug delivery system including a rivastigmine-containing drug layer; a supporter adhered to one surface of the drug layer to support the drug layer; and a release film or release paper adhered to the other surface of the drug layer, wherein the drug layer contains 10 to 40 parts by weight of a rubber, 20 to 80 parts by weight of a rosin ester resin and 0.1 to 10 parts by weight of an acrylic adhesive, and the drug layer has a thickness of 40 μm to 100 μm.

Preferably, the acrylic adhesive contains a carboxyl group.

Preferably, the rubber includes natural rubber, isoprene rubber, polyisobutylene, a styrene-butadiene block copolymer, a styrene-butadiene-styrene block copolymer, a styrene-isoprene-styrene block copolymer or a combination thereof.

Preferably, the rosin ester resin includes rosin glycerin ester, hydrogenated rosin glycerin ester, rosin pentaerythritol ester or a combination thereof.

FIG. 1 illustrates a tri-layer transepidermal drug delivery system containing rivastigmine according to the present invention. The tri-layer transepidermal drug delivery system according to the present invention exhibits drug dissolution and skin permeation effects comparable to commercially available control drug E and causes almost no irritation of the skin. In addition, the drug layer is fixed to the skin due to superior adhesivity thereof, is not detached from the skin and absorbed in the skin at a constant rate, although it is adhered to the skin for a long period of time. Accordingly, the transepidermal drug delivery system is considerably useful for the treatment of Alzheimer's disease and Parkinson's disease-type dementia.

Referring to FIG. 1, the transepidermal drug delivery system according to the present invention includes three layers to allow the drug to be effectively absorbed in the skin. Specifically, the transepidermal drug delivery system of the present invention includes a drug layer 1, a supporter 2 and a release film or a release paper 3.

In the present invention, the drug layer 1 directly contacts the skin and controls drug dissolution and skin permeation rate. The drug layer contains rivastigmine, a natural or synthetic rubber, a rosin ester resin and an acrylic adhesive. The rivastigmine may be used in itself or as a salt thereof. In addition, the rivastigmine is present in a state of being dissolved in the drug layer, and the content of rivastigmine is about 18 mg per a drug layer area of 10 cm², but is not limited thereto. In addition, distinctively, the drug layer of the present invention does not contain a transdermal absorption accelerator. As used herein, the term “about” means presence within a predetermined range of the corresponding value (for example, within ±5%, or within ±10%). As such, rivastigmine may be present in an amount of 15 to 50% by weight (or parts by weight) in the drug layer.

The drug layer excluding rivastigmine may contain a rubber, a rosin ester resin and an acrylic adhesive.

The rubber according to the present invention may be used in an amount of 10 to 40 parts by weight, preferably 15 to 30 parts by weight in order to control the strength of the drug layer. When the rubber is used in an amount lower than 10 parts by weight, the strength of the drug layer decreases and when the rubber is used in an amount higher than 40 parts by weight, strength increases, adhesivity decreases and flexibility decreases. The rubber according to the present invention includes natural rubber, synthetic rubber or a mixture thereof, preferably natural rubber, isoprene rubber, polyisobutylene, a styrene-butadiene block copolymer, a styrene-butadiene-styrene block copolymer, a styrene-isoprene-styrene block copolymer or a mixture thereof.

In the present invention, the rosin ester resin may be used as an adhesion-applying agent in an amount of 20 to 80 parts by weight, preferably 30 to 60 parts by weight. When the rosin ester resin is used in an amount lower than 20 parts by weight, it is difficult to adhere the transepidermal drug delivery system to the skin due to low adhesivity. In addition, when the rosin ester resin is used in an amount exceeding 80 parts by weight, disadvantageously, the drug layer is excessively sticky and sticky residues are left behind upon removal from the skin. In the present invention, the rosin ester resin preferably contains rosin glycerin ester, hydrogenated rosin glycerin ester, rosin pentaerythritol ester or a mixture thereof. More preferably, the rosin ester resin contains hydrogenated rosin glycerin ester or a mixture thereof.

In the present invention, the acrylic adhesive may be used in an amount of 0.1 to 10 parts by weight, preferably 3 to 7 parts by weight. When the acrylic adhesive is used in an amount lower than 0.1 parts by weight, the amount of transdermally absorbed rivastigmine exceeds the level comparable to the control drug E. When the acrylic adhesive is used in an amount higher than 10 parts by weight, the amount of rivastigmine transdermally absorbed is decreased below a level comparable to the control drug E and aggregation between the acrylic adhesive and the adhesive material occurs, thus making it impossible to uniformly manufacture the drug layer.

Preferably, in the present invention, the acrylic adhesive contains a carboxyl group. The carboxyl group of the acrylic adhesive interacts with rivastigmine to suitably control drug release. Accordingly, the acrylic adhesive according to the present invention may be used to control the amount of transdermally absorbed rivastigmine and to apply adhesion.

In the present invention, the acrylic adhesive comprises a (co)polymer having one or more monomers selected from acrylate (alkyl acrylate) and alkyl methacrylate, without being limited thereto. In addition, the acrylic adhesive comprises a (co)polymer having one or more monomers selected from C4˜C17 acrylate (alkyl acrylate) and C4˜C17 alkyl methacrylate. In addition, preferably, the acrylic adhesive contains a (co)polymer having one or more monomers selected from 2-ethylhexyl acrylate (2-EHA), butyl acrylate (BA), ethyl acrylate (EA) and iso-octyl acrylate (iso-OA). In addition, preferably, the acrylic adhesive contains a (co)polymer containing 2-ethylhexyl acrylate.

In the present invention, the drug layer has a thickness of 40 to 100 μm, preferably 50 to 90 μm. When the thickness of drug layer is lower than 40 μm, the concentration of drug contained in the drug layer increases, and dissolution and transdermal absorptivity of the drug increase. In addition, when the thickness of the drug layer is higher than 100 μm, the concentration of drug contained in the drug layer decreases, and dissolution and transdermal absorptivity of the drug thus decrease.

The supporter 2 used in the present invention supports the drug layer, prevents contamination by foreign materials when adhered, and allows the drug to be administered to the skin to prevent reverse diffusion of the drug. The supporter includes a polyester film on which transparent, semi-transparent or non-transparent aluminum is deposited, a polyester film on which non-woven fabric is laminated, a polyethylene terephthalate film or a combination thereof. Preferably, the supporter has a thickness of 5 μm to 50 μm. When the thickness is lower than 5 μm, the supporter is non-uniformly laminated to the drug layer during manufacture due to high flexibility thereof. On the other hand, when the thickness is 50 μm or higher, the delivery system may be readily detached from the skin during movement due to excessively low flexibility of the delivery system, although adhesivity of drug layer is high.

The release film or release paper 3 according to the present invention is adhered to the other surface of the drug layer, protects the drug layer from foreign materials and is readily separated from the drug layer when the delivery system is used. The configuration of the release film or release paper is exemplified in FIG. 2. The release film may be a film composed of polyester, polyvinyl chloride, polyvinylidene chloride, polyethylene terephthalate or the like. In addition, the release paper may be paper such as polyolefin-laminated wood free paper or glassine paper. The surface of the release film or release paper which contacts the drug layer may be coated with a silicone or fluorine resin so that the delivery system is readily separated from the drug layer. Preferably, the release film or release paper includes a semi-transparent polyethylene terephthalate release film with a thickness of about 75 μm in which one surface of the release film or release paper is coated with a silicone resin.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide further understanding of the disclosure and are incorporated in and constitute a part of this application, illustrate embodiments of the disclosure and together with the description serve to explain the principle of the disclosure. In the drawings:

FIG. 1 is a sectional view illustrating a patch-type transepidermal drug delivery system according to one embodiment of the present invention;

FIG. 2 is a plan view illustrating a release film or release paper in the transepidermal drug delivery system;

FIG. 3 shows relative dissolution test results of Examples 1, 3 and 4, Comparative Examples 1 and 2 and control drug E to compare drug dissolution behaviors depending on the content of acrylic adhesive;

FIG. 4 shows relative dissolution test results of Examples 2 and 5, Comparative Examples 5 and 6 and control drug E to compare drug dissolution behaviors depending on the drug layer thickness;

FIG. 5 shows transdermal permeation test results of Examples 1, 3 and 4, Comparative Examples 1 and 2 and control drug E to compare skin permeation behaviors depending on content of acrylic adhesive; and

FIG. 6 shows transdermal permeation test results of Examples 2 and 5, Comparative Examples 5 and 6 and control drug E to compare skin permeation behaviors depending on drug layer thickness.

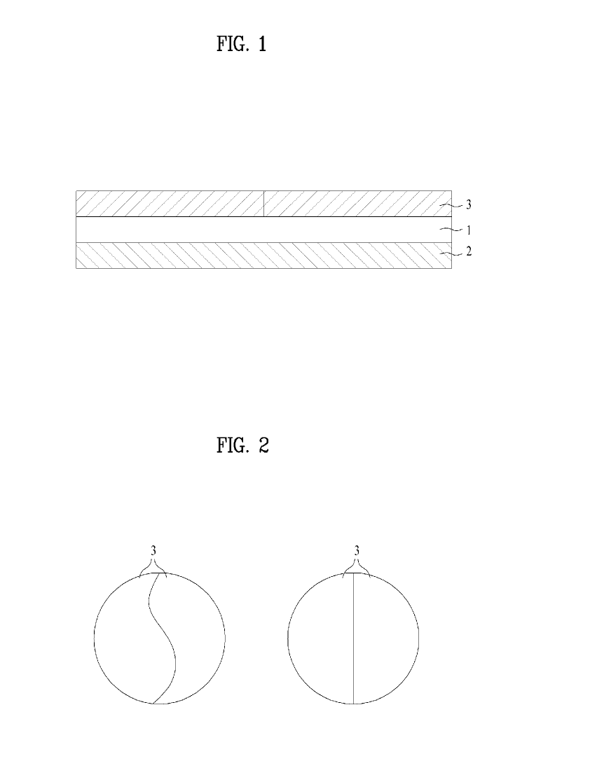

### DETAILED DESCRIPTION OF THE INVENTION

Reference will now be made in detail to the specific embodiments of the present invention, examples of which are illustrated in the accompanying drawings.

Now, the present invention will be described in more detail with reference to the following Examples. These examples are only provided to illustrate the present invention and should not be construed as limiting the scope and spirit of the present invention.

Minimum examples are selected from a variety of repeated tests in accordance with Examples and Comparative Examples for better understanding of the present invention. The addition order of respective components in the following Examples is provided as one example and is not limited thereto.

### Example 1

30% by weight of rivastigmine, 23.3% by weight of natural rubber, 46.6% by weight of hydrogenated rosin glycerin ester and 0.1% by weight of an acrylic adhesive (containing a carboxyl group-linked 2-EHA monomer) were dissolved in a solution in which a ratio of toluene to heptane was 7:3 (wt/wt). Here, the weight is based on dry weight. The resulting solution was applied to the silicone resin-treated surface of a semi-transparent polyethylene terephthalate release film with a thickness of 75 μm, followed by drying, to produce a drug layer with a thickness of 70 μm. The produced drug layer was transferred to a semi-transparent polyethylene terephthalate film supporter with a thickness of 20 μm thickness and laminated thereto. Then, the laminate was cut to a predetermined size of 10 cm² with a cutter.

### Examples 2 to 5 and Comparative Examples 1 to 6

Examples 2 to 5 and Comparative Examples 1 to 6 were produced in the same manner as in Example 1 in accordance with different compositions. Table 1 shows compositions of Examples 2 to 5 and Comparative Examples 1 to 6. Commercially available Exelon Patch 10 (18 mg) was used as a control drug E.

TABLE 1

| | Ex. | Ex. | Ex. | Ex. | Comp. | Comp. | Comp. | Comp. | Comp. | Comp. |
|---|---|---|---|---|---|---|---|---|---|---|
| Items | 2 | 3 | 4 | 5 | Ex. 1 | Ex. 2 | Ex. 3 | Ex. 4 | Ex. 5 | Ex. 6 |
| Rivastigmine | 45 | 25.71 | 25.71 | 18 | 25.71 | 25.71 | 18 | 18 | 51.43 | 17.14 |
| Natural | 16.67 | 23.10 | 21.43 | 24 | 24.76 | 21.40 | 0.50 | 80.50 | 14.52 | 25.95 |
| rubber | | | | | | | | | | |
| Hydrogenated | 33.33 | 46.19 | 42.86 | 48 | 49.53 | 42.79 | 80.50 | 0.50 | 29.05 | 51.91 |
| rosin glycerin | | | | | | | | | | |
| ester | | | | | | | | | | |
| Acrylic | 5 | 5 | 10 | 10 | 0 | 10.10 | 1 | 1 | 5 | 5 |
| adhesive | | | | | | | | | | |
| Total | 100 | 100 | 100 | 100 | 100 | 100 | 100 | 100 | 100 | 100 |
| Thickness of | 40 μm | 70 μm | 70 μm | 100 μm | 70 μm | 70 μm | 100 μm | 100 μm | 35 μm | 105 μm |
| drug layer | | | | | | | | | | |

Based on wt %

### Test Examples

Skin irritation level testing, adhesivity testing, relative dissolution testing and transdermal permeation testing were performed to compare qualities between the transepidermal drug delivery systems produced in accordance with Examples 1 to 5 and Comparative Examples 1 to 6 with Exelon Patch 10 (control drug E) and evaluate the same.

### Test Example 1

### Skin Irritation Level Test

The samples (having a circular shape with a size of 10 cm²) of Examples 1 to 5, Comparative Examples 1 to 6 and the control drug E were attached to the inside of the antebrachium of both arms of 20 subjects for 24 hours. The samples were separated from the antebrachium and erythema was then evaluated by the naked eye for 30 minutes in accordance with evaluation grades as set forth in Table 2.

TABLE 2

| | | Almost no | | Weak | |
|---|---|---|---|---|---|
| | No | irritation | Medium | irritation | irritation |
| Item | irritation (−−) | (−) | (+−) | (+) | (++) |
| Evaluation | 5.0 | 4.0 | 3.0 | 2.0 | 1.0 |
| grade | | | | | |

### Test Example 2

### Adhesivity Test

Examples 1 to 5, Comparative Example 1 and control drug E were subjected to adhesivity tests using a tensile strength meter. The tensile strength testing was carried out by the following method. A sample was cut to a width of 12 mm and adhered to one edge of a phenol resin test plate (width: 25 mm, length: 125 mm, thickness: 5 mm) which had been allowed to stand in a constant temperature and humidity chamber at a relative humidity of 65±5% and temperature of 37° C. for 30 minutes. The sample was passed through an 850 g rubber roller at a rate of 300 mm/min twice and the sample-adhered test plate was allowed to stand in a constant temperature and humidity chamber for 30 minutes. The edge of the test plate-adhered sample was bent backward by an angle of 180 degrees and fixed on an upper part of tensile strength meter, and the test plate was fixed to a lower part thereof. The sample was drawn at a rate of 300 mm/min to a distance of 20 mm. At this time, loads were measured 10 times and an average of the 10 values was then recorded.

### Test Example 3

### Relative Dissolution Test

6 samples (10 cm²/one) for each of Examples 1 to 5, Comparative Examples 1, 2, 5 and 6 and control drug E were adhered to a double-sided adhesive tape-attached disc assembly such that the drug layer was directed upward. 500 ml of water was added to a dissolution vessel at a dissolution temperature of 32±0.5° C. and 1 mL of the dissolution solution was collected at a paddle rotation rate of 50 rpm for 30 minutes, 4 hours and 24 hours. Rivastigmine contained in the collected dissolution solution was analyzed by HPLC. HPLC analysis conditions were as follows. A solution consisting of pH 3.0 phosphate buffer:acetonitrile (70:30 (v/v)) was used as a mobile phase, injected amount was 10 μl, flow rate was 1 mL/mn, detection wavelength was 217 nm and Agilent Zorbox XDB C18 was used as a column. The similarity was evaluated in accordance with a paddle over disk method of United States Pharmacopeia (USP) transepidermal drug delivery system.

### Test Example 4

### Transdermal Permeation Test

Examples 1 to 5, Comparative Examples 1, 2, 5 and 6 and the control drug E were subjected to transdermal permeation tests. The cervical vertebrae of a 6-week old male hairless mouse was dislocated, the skin was cut to a size of 3 cm×3 cm and subcutaneous fat was removed. Circular-shaped samples (cut to a size of 1.767 cm²) of Examples 1 to 5, Comparative Examples 1, 2, 5 and 6 and control drug E were cut and adhered to the epidermal layer. The endodermal layer of the skin was adhered such that it faced Franz diffusion cells. PBS (Phosphate buffered saline, pH 7.4) was carefully filled in a receptor layer of the Franz diffusion cells such that foaming did not occur and stirred at 600 rpm while maintaining the temperature of a Franz diffusion apparatus at 32±0.5° C. 300 μl of each sample was collected at 30 minutes and 1, 2, 3, 4, 5, 6, 8, 10, 12 and 24 hours and the content of rivastigmine was assayed by HPLC analysis and the equivalent amount of buffer solution was filled in the receptor.

The active skin area of the hairless mouse adhered to the Franz diffusion cell was 1.767 cm², the receptor volume was 13.0 ml, and skin permeation tests were performed under sink conditions. HPLC analysis conditions were the same as HPLC analysis conditions of relative dissolution tests.

Test Results

The results of skin irritation level tests are shown in Table 3. It could be seen that Examples 1 to 5, Comparative Example 1 and control drug E caused almost no skin irritation. Comparative Example 2 caused erythema during removal due to strong adhesion caused by high content of acrylic adhesive, and Comparative Examples 3, 5 and 6 caused erythema during removal of the remaining drug layer using an alcohol gauze due to higher hydrogenated rosin ester glycerin content, higher rivastigmine content and drug layer thickness, as compared to other components. Comparative Example 4 could not exhibit effective results since hydrogenated rosin ester glycerin to provide adhesivity is present in an excessively low amount and the drug layer was not sufficiently adhered to the skin.

TABLE 3

| Test | Examples | | | | | Comparative Examples | | | | | | |
|---|---|---|---|---|---|---|---|---|---|---|---|---|
| Examples | 1 | 2 | 3 | 4 | 5 | 1 | 2 | 3 | 4 | 5 | 6 | E |
| Average | 4.90 | 4.80 | 4.92 | 4.80 | 4.90 | 4.74 | 2.50 | 2.64 | — | 2.56 | 2.80 | 4.9 |

The adhesivity test results are shown in Table 4. The adhesivity of the transepidermal drug delivery system is considerably important for stable drug delivery through the skin. Comparative Examples 2, 3, 5 and 6 which exhibits serious skin irritation, and Comparative Example 4 which exhibits considerably decreased skin adhesivity, based on the skin irritation level tests, were excluded in this test. Adhesivity was measured using the tensile strength meter. As a result, it could be seen that Examples 1 to 5 containing an acrylic adhesive exhibited higher adhesivity, as compared to the control drug E, when comparing Examples 1, 3 and 4, and as the content of the acrylic adhesive increased, adhesivity was improved. When comparing Examples 2, 3 and 4 with Example 5, in a case where the samples contain the same content of acrylic adhesive, as the thickness increased, adhesivity was improved. Comparative Example 1 containing no acrylic adhesive exhibited relatively low adhesivity.

TABLE 4

| | Comp. | Control | | | | | |
|---|---|---|---|---|---|---|---|
| Item | Ex. 1 | drug E | Ex. 1 | Ex. 2 | Ex. 3 | Ex. 4 | Ex. 5 |
| Adhesitvity | 102.65 ± | 403.89 ± | 417.24 ± | 423.42 ± | 445.31 ± | 468.53 ± | 489.35 ± |
| (gf) | 9.04 | 5.24 | 8.34 | 4.53 | 9.85 | 7.53 | 8.51 |

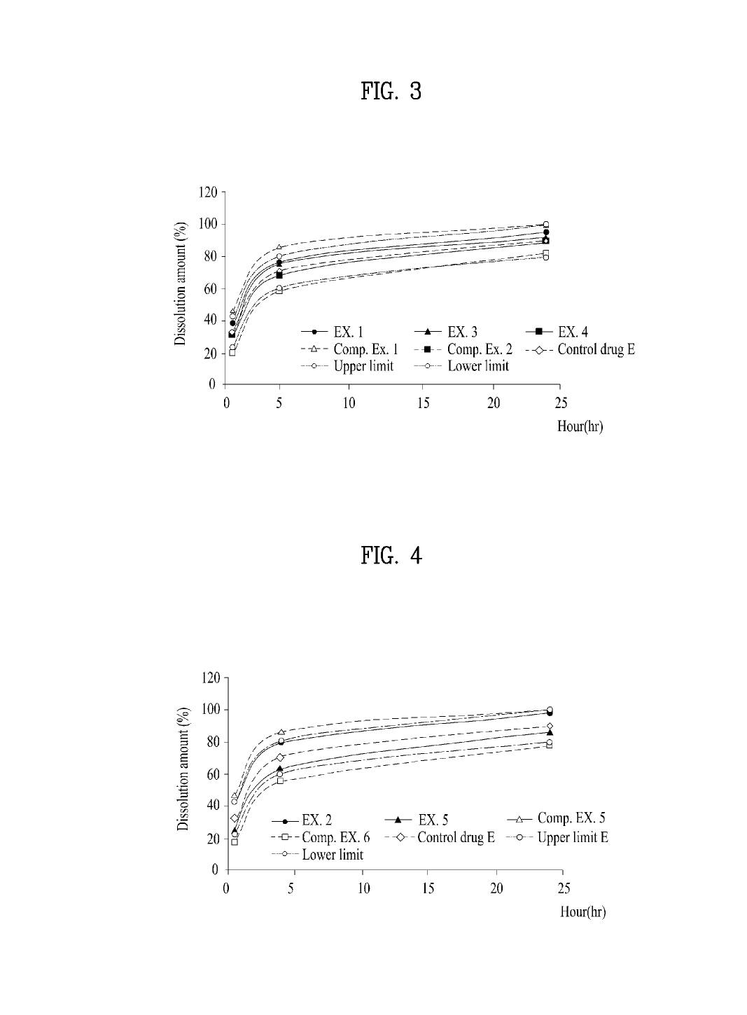

The relative dissolution and transdermal permeation tests are good test methods to efficiently determine equivalence to the control drug regarding efficacies and effects of the present invention. Except Comparative Example and Comparative Example 4 which are unsuitable for development of drug systems, based on skin irritation level and adhesivity test results, Examples 1 to 5, Comparative Examples 1, 2, 5 and 6 and control drug E were subjected to relative dissolution testing. The results thus obtained are shown in FIGS. 3 and 4. FIGS. 3 and 4 show drug dissolution behaviors in accordance with acrylic adhesive content and drug layer thickness, respectively. The relative dissolution test is considerably important in confirming drug equivalence to the control drug. When an average dissolution rate of a test drug is within ±10% of the average dissolution rate of a control drug, the test drug is considered to be equivalent to the control group by Food & Drug Administration. When comparing the average dissolution between the drug of Example with control drug E via a relative dissolution test, average dissolution rates of Examples 1 to 5 are within an equivalence range of ±10%, which indicates that the drug of Example exhibits efficacies comparable to the control drug. The composition suggested by the present invention is considered to be a composition which exhibits efficacies comparable to the control drug. It could be seen that the average dissolution rates of Comparative Examples 1, 2, 5 and 6 are out of the equivalence range.

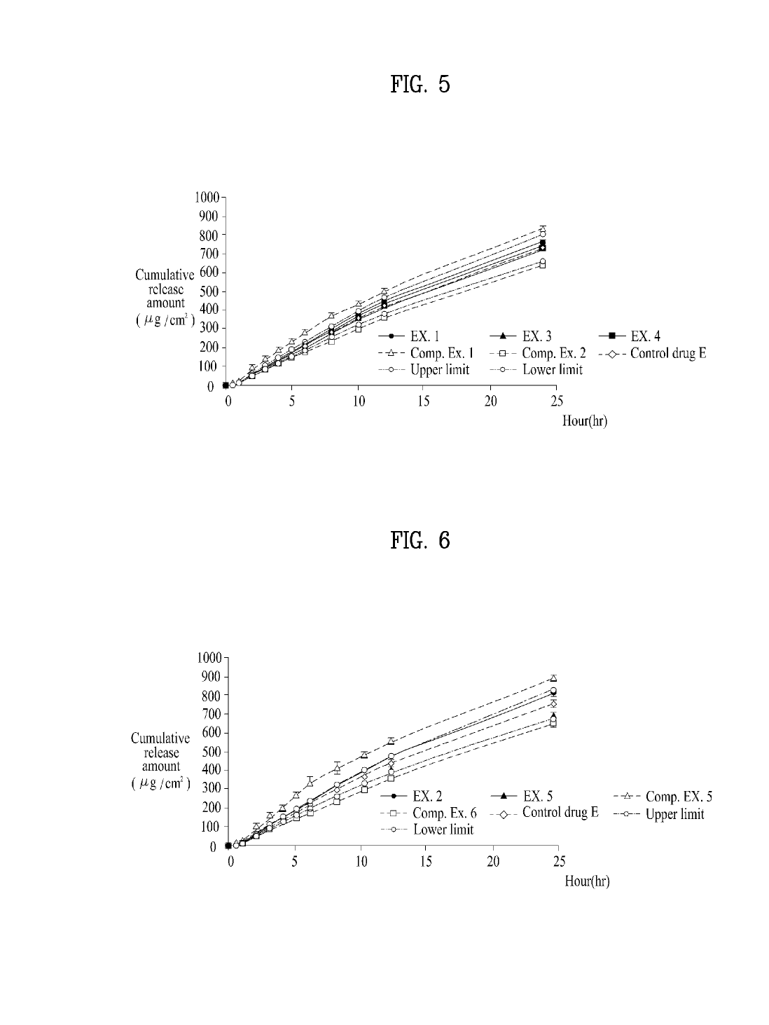

The transdermal permeation test was performed on Examples 1 to 5, Comparative Examples 1, 2, 5 and 6 and control drug E which underwent the relative dissolution test. The transdermal permeation test results are shown in FIGS. 5 and 6. Referring to FIGS. 5 and 6, skin permeation behaviors in accordance with the acrylic adhesive content and drug layer thickness are shown. The transdermal permeation test results are similar to relative dissolution test results. In accordance with the relative dissolution test and transdermal permeation test results, in Examples 1, 3 and 4, as the content of acrylic adhesive increases, linkage level of the carboxyl group of the acrylic adhesive to the amine group of rivastigmine increases and drug release can thus be suitably controlled. In accordance with the results of Example 2 and Example 5, as the thickness of the drug layer increases, drug dissolution decreases. Comparative Examples 1 and 5 exhibited rapid drug dissolution behaviors since they did not contain an acrylic adhesive or had an excessively small drug layer thickness, while Comparative Examples 2 and 6 exhibited considerably deteriorated drug dissolution, since they contained excess acrylic adhesive or had an excessively large drug layer thickness.

The present invention provides a transepidermal drug delivery system which contains rivastigmine, a rubber, a rosin ester resin and an acrylic adhesive-containing drug layer. In addition, the present invention provides a transepidermal drug delivery system which a three-layer structure including a drug layer, a supporter, and a release film or a release paper. The transepidermal drug delivery system of the present invention exhibits drug dissolution and skin permeation effects comparable to commercially available control drug E. In addition, the drug layer which directly contacts the skin is treated with a component harmless to the human body and thus causes almost no skin irritation. In addition, the transepidermal drug delivery system is useful for treatment of Alzheimer's disease and Parkinson's disease-type dementia, since the drug layer having superior adhesivity is fixed to the skin, the drug layer is not detached from the skin, and the drug is absorbed in the skin at a predetermined rate, although the delivery system is adhered to the skin for a long period of time.

As apparent from the results, the present invention is applicable to pharmaceutical compositions containing rivastigmine. Specifically, the present invention is applicable to transepidermal drug delivery systems containing rivastigmine for treating Alzheimer's disease and Parkinson's disease-type dementia.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A transepidermal drug delivery system comprising:
a rivastigmine-containing drug layer; and
a supporter adhered to one surface of the drug layer to support the drug layer,
wherein the drug layer contains 10 to 40 parts by weight of a rubber, 20 to 80 parts by weight of a rosin ester resin and 0.1 to 10 parts by weight of an acrylic adhesive, and the drug layer has a thickness of 40 μm to 100 μm.

2. The transepidermal drug delivery system according to claim 1, further comprising: a release film or release paper adhered to the other surface of the drug layer,
wherein the transepidermal drug delivery system has a three-layer structure.

3. The transepidermal drug delivery system according to claim 1, wherein the acrylic adhesive has a carboxyl group.

4. The transepidermal drug delivery system according to claim 1, wherein the rubber comprises natural rubber, isoprene rubber, polyisobutylene, a styrene-butadiene block copolymer, a styrene-butadiene-styrene block copolymer, a styrene-isoprene-styrene block copolymer or a combination thereof.

5. The transepidermal drug delivery system according to claim 1, wherein the rosin ester resin comprises rosin glycerin ester, a hydrogenated rosin glycerin ester, a rosin pentaerythritol ester or a combination thereof.
